(19) United States
(12) Patent Application Publication
(10) Pub. No.: US 2015/0359562 A1
(43) Pub. Date: Dec. 17, 2015

(54) ARTHROSCOPIC JOINT SURGERY DEVICE AND METHOD OF USE

(71) Applicant: Richard L. Needleman, Plymouth, MI (US)

(72) Inventor: Richard L. Needleman, Plymouth, MI (US)

(21) Appl. No.: 14/305,341

(22) Filed: Jun. 16, 2014

(51) Int. Cl. A61B 17/34; A61B 17/00

(52) U.S. Cl. CPC A61B 17/3423; A61B 17/00234; A61B 17/3415; A61B 2017/3456

(57) ABSTRACT

An arthroscopic joint surgery device and method of use. The device has a tube and a knob with a hole that has an inner diameter matching the inner diameter of the tube and mates with the inner diameter of the tube. The tube and knob can be permanently attached or the knob can be configured to enable the knob to be unattached from the tube. The utilization of the device in this method enables surgeons to safely and efficiently locate the joint portals for the placement of the arthroscope and for a separate inflow portal. The arthroscope is in the proper position to visualize the whole joint. Damage to the joint surface cartilage is minimized. After needle localization of the joint under fluoroscopic guidance, the method uses the device to dilate a joint portal opening, such as an anteromedial ankle portal in preparation for the placement of the arthroscope or the posterolateral portal for the introduction of the inflow cannula. The method is performed under fluoroscopic guidance.

### TECHNICAL FIELD

The present disclosure relates to the field of arthroscopic trocar or obturator devices for joint surgery and their methods of use. More particularly, the disclosure discusses devices and methods for smaller joints such as an ankle.

### BACKGROUND

Trocar and obturator devices are currently successfully used for arthroscopic knee and shoulder surgery. These joints have a thin layer of overlying soft tissue so that the surgeon can identify the external anatomic landmarks and develop small portals into the joints. The hip had been a difficult joint to arthroscope because it is a deep joint with a thick layer of overlying soft tissues that were not amenable to palpating external anatomic landmarks accurately, especially in obese patients. Recently, there has been a development of arthroscopic hip surgery by using external anatomic topography, noninvasive distraction, and fluoroscopy. Modifications have been made to the basic knee and shoulder arthroscopic instruments to account for differences in the joint size and for the localization of the hip joint with fluoroscopy. The hip instruments are larger and the trocars and obturators have been cannulated to allow for the successive passing of larger dilators into the hip joint.

A trocar has a sharp end for introduction into the joint. In contrast, an obturator has a blunt end for introduction into the joint. A surgeon chooses the device that they wish to use. The sharp-ended trocar can scuff and damage a patient's joint cartilage. However, a blunt-ended obturator may not be able to penetrate a thick joint capsule.

### BRIEF SUMMARY OF THE INVENTION

In one embodiment the arthroscopic joint surgery device comprises: a tube measuring approximately 2.7 mm outer diameter, approximately 1.2 mm inner diameter, and approximately 85 mm length, and having a blunt rounded distal end and a blunt rounded proximal end; and a cannulated blunt knob measuring approximately 15 mm outer diameter, approximately 1.2 mm inner diameter, and approximately 2.4 mm length, wherein the knob is attached to the proximal end of the tube.

The tube can be constructed of metal or the like, so that it is easily sterilized. In a separate embodiment, the tube is a one-use disposable element constructed of metal, plastic, or the like.

The attachment between the tube and the knob can be a threaded connection or the like. In this embodiment, the tube would have male threads and the knob would have female threads. Alternately, the knob can be permanently attached to the tube (e.g. welded or the like).

In one embodiment, the knob can be truncated and ridged for easy handling by a surgeon.

For the purposes of this disclosure, an angiocatheter is a device that can be inserted into a blood vessel, usually a vein. Medications, hydrating fluid, and/or products such as blood can be administered through this access port into the blood system. A metal stylet is within a cannulated flexible plastic tube. The metal stylet has a sharp point to allow for penetration of the skin and soft structures between the skin and the vein. It also allows for penetration into the vein. It is inserted into a vein with the metal stylet in to keep it rigid. This is important during insertion. Once the catheter is within the vein, the stylet is removed (and the plastic catheter is advanced further into the vein) so that medications, hydrating fluid, and/or products such as blood can be administered through this access port into the blood system. Meanwhile, the catheter becomes flexible that allows for less trauma and pain while it remains within the blood vessel.

For the purposes of this disclosure, a cannulated needle is a device that can be inserted near the spinal cord. It is called a ‘spinal needle’. Medication can be administered or a specimen of spinal fluid can be obtained through this access port. A metal stylet is within a rigid metal tube. The metal stylet has a sharp point to allow for penetration of the skin and soft structures between the skin and the spinal cord. It is inserted near the spinal cord with the metal stylet in. Once the needle is in the proper position next to the spinal cord, the stylet is removed so that medication can be administered or a specimen of spinal fluid can be obtained through this access port. The needle remains rigid which allows for it to remain in the appropriate position in the short time that the procedure is performed.

A method to use the arthroscopic joint surgery device described above comprises: inserting an angiocatheter with a stylet or a cannulated needle with a stylet into a joint portal under fluoroscopic guidance; pulling the stylet out of the angiocatheter or cannulated needle and inserting a guide-wire into the angiocatheter or needle under fluoroscopic guidance; removing the angiocatheter or cannulated needle; sliding the distal end of the tube over the guide-wire and then pushing the device into the joint portal under fluoroscopic guidance; detaching the knob from the tube; removing the knob and the guide-wire; sliding an arthroscopic sheath over the tube into the joint portal under fluoroscopic guidance; pulling out the tube; inserting an arthroscope into the arthroscopic sheath under fluoroscopic guidance; and beginning an arthroscopic procedure.

In a separate embodiment, the knob is permanently attached to the tube. Hence the steps are modified, the method comprising: inserting an angiocatheter with a stylet or a cannulated needle with a stylet into a joint portal under fluoroscopic guidance; pulling the stylet out of the angiocatheter or cannulated needle and inserting a guide-wire into the angiocatheter or needle under fluoroscopic guidance; removing the angiocatheter or cannulated needle; placing the device into an arthroscopic sheath; sliding the distal end of the tube and arthroscopic sheath over the guide-wire into the joint portal; removing the guide-wire; and beginning an arthroscopic procedure.

A joint can be an ankle, knee, wrist, or the like.

For an ankle joint embodiment, the joint portal can be the anteromedial portal, anterolateral portal, posterolateral portal, or the like.

Fluoroscopy is the use of x-rays in real-time. It allows for the visualization of radiopaque structures such as the ankle bones and radiopaque devices such as an angiocatheter with a stylet or needle with a stylet, the arthroscopic joint surgery device or tube, the arthroscopic sheath, and the arthroscope. Fluoroscopic guidance is used to verify that these radiopaque devices are within the joint that is between two adjacent bones. These images are displayed on a monitor. Water based fluids (such as joint irrigating fluid and blood) are radiolucent and are not visualized by x-rays.

The scope of the invention is defined by the claims, which are incorporated into this section by reference. A more complete understanding of embodiments on the present disclosure will be afforded to those skilled in the art, as well as the realization of additional advantages thereof, by consideration of the following detailed description of one or more embodiments. Reference will be made to the appended sheets of drawings that will first be described briefly.

### BRIEF DESCRIPTION OF THE DRAWINGS

FIG. 1 is a flow diagram of a method to use the arthroscopic joint surgery device.

FIG. 2 shows a stylet.

FIG. 3 shows an angiocatheter.

FIG. 4 shows a stylet in an angiocatheter.

FIG. 5 shows a guide-wire.

FIG. 6 shows a perspective view of an arthroscopic joint surgery device.

FIG. 7 shows a guide-wire within an arthroscopic joint surgery device.

FIG. 8 shows the primary ankle bones.

FIG. 9 shows two joint portals, the ‘working’ portal is the anterolateral portal and the ‘viewing’ portal is the anteromedial portal.

FIG. 10 shows needle localization of the ankle joint via the anteromedial portal.

FIG. 11 shows angiocatheter localization of the ankle joint via the anteromedial portal.

FIG. 12 shows the stylet removed.

FIG. 13 shows a guide-wire in the anteromedial portal after the angiocatheter or the cannulated needle has been removed.

FIG. 14 shows an arthroscopic sheath.

FIG. 15 shows a guide wire inside an arthroscopic joint surgery device that is inside an arthroscopic sheath.

FIG. 16 shows a cannulated needle.

FIG. 17 shows a stylet inserted in a cannulated needle and not secured to the hub of the needle.

FIG. 18 shows a perspective view of a standard obturator.

### DETAILED DESCRIPTION OF THE INVENTION

The present disclosure discusses an arthroscopic joint surgery device and method of use. The device and method of use enable improved surgery techniques in medium and small joints such as ankle joints. Previously, these improved surgery techniques were only available in larger joints such as the hip joint.

The method is an adaptation of common hip surgery techniques to small joint arthroscopy by performing the procedure under the guidance of fluoroscopy, placing an angiocatheter with a stylet or a cannulated needle with a stylet into the joint portal prior to dilation, placing a blunt guide-wire into the angiocatheter or cannulated needle, and using the device as described herein to dilate the joint portal opening prior to inserting an arthroscopic sheath.

The soft issues overlying the anterior ankle are thin and the anatomic landmarks for portal placement are usually palpable. Oftentimes under pathologic conditions, the ankle joint is swollen with edema and consequently the anterior landmarks can be difficult to palpate. The soft tissues overlying the posterior ankle are thick and the anatomic landmarks for portal placement are difficult to palpate. The access of posterior portals is difficult and not attempted by most surgeons. Therefore, most surgeons usually do not have the optimum numbers of portals for the performance of arthroscopic ankle surgery.

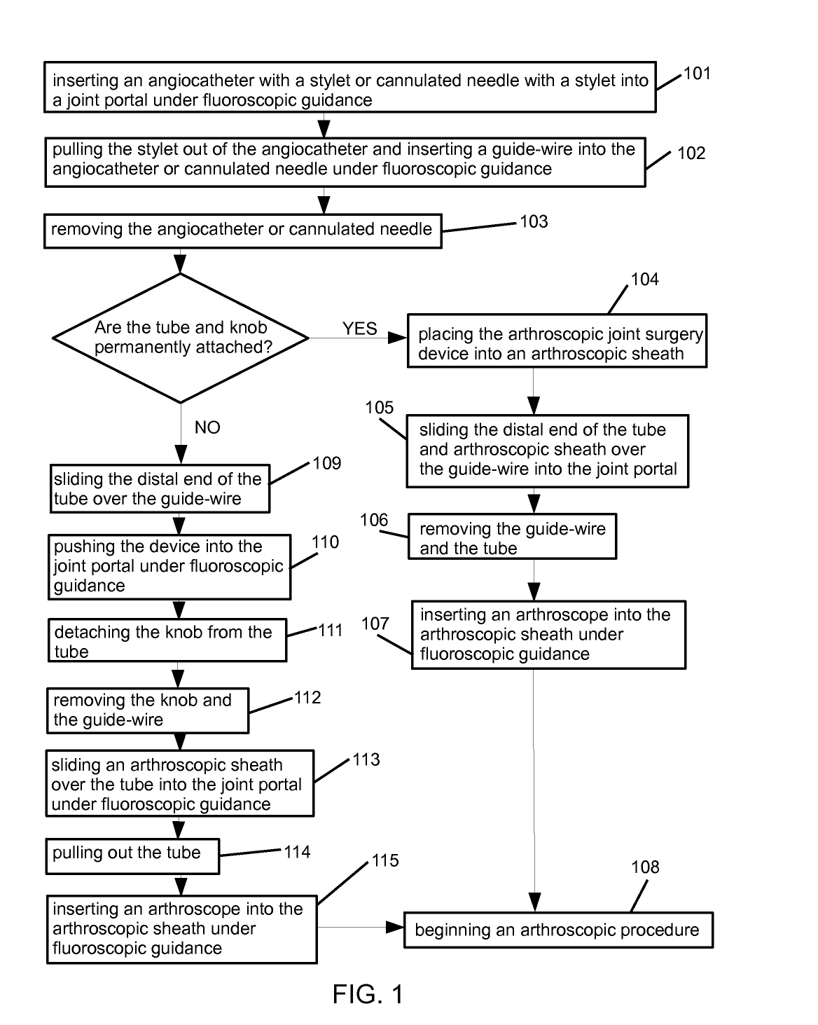

FIG. 1 is a flow diagram of a method to use the arthroscopic joint surgery device.

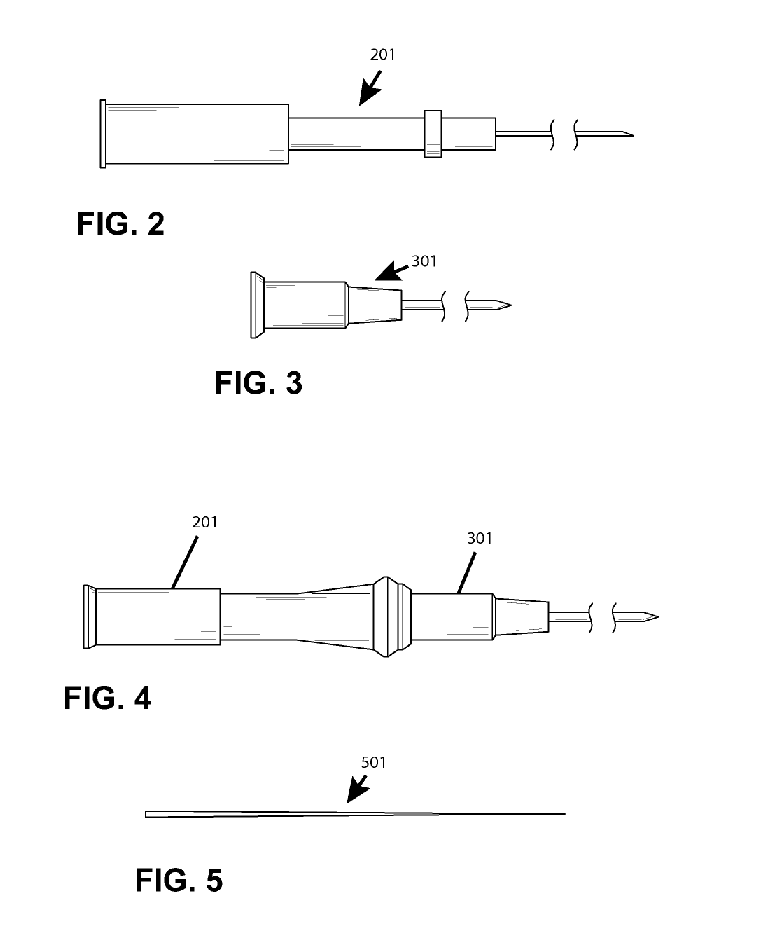

FIG. 2 shows a stylet. Shown is stylet 201.

FIG. 3 shows an angiocatheter. Shown is angiocatheter 301.

FIG. 4 shows a stylet inserted in an angiocatheter. Shown are stylet 201 and angiocatheter 301.

FIG. 5 shows a guidewire 501.

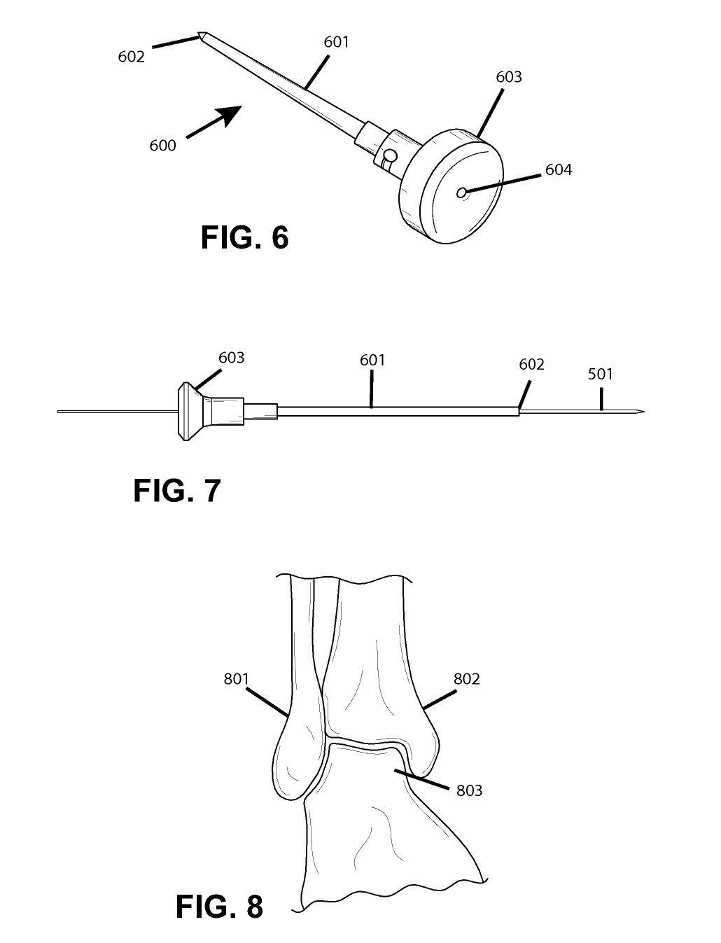

FIG. 6 shows a perspective view of an arthroscopic joint surgery device. Shown are the device 600, a tube 601 with a distal end 602, a knob 603, and a hole in the knob 604 (that extends to the tube and matches the tube inner diameter).

FIG. 7 shows a guide-wire inserted within an arthroscopic joint surgery device. Shown are a tube 601 with a distal end 602, a knob 603, and a guide-wire 501.

FIG. 8 shows the primary ankle bones. Shown are the fibula 801, tibia 802, and talus 803.

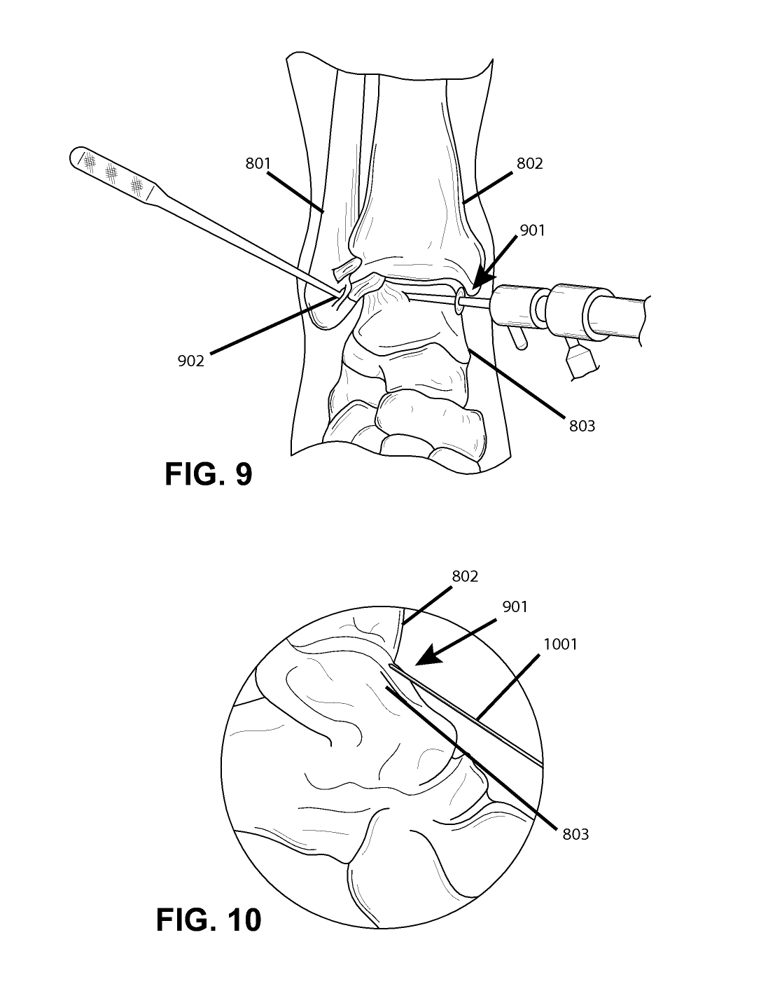

FIG. 9 shows two joint portals, the ‘working’ portal is the anterolateral portal and the ‘viewing’ portal is the anteromedial portal. Shown are the anteromedial portal 901, fibula 801, tibia 802, talus 803, and anterolateral portal 902.

FIG. 10 shows needle localization. Shown are a needle 1001, anteromedial portal 901, tibia 802, and talus 803. The drawing shows a typical view under fluoroscopic conditions. The needle 1001 is visible but the non-metallic element is not visible.

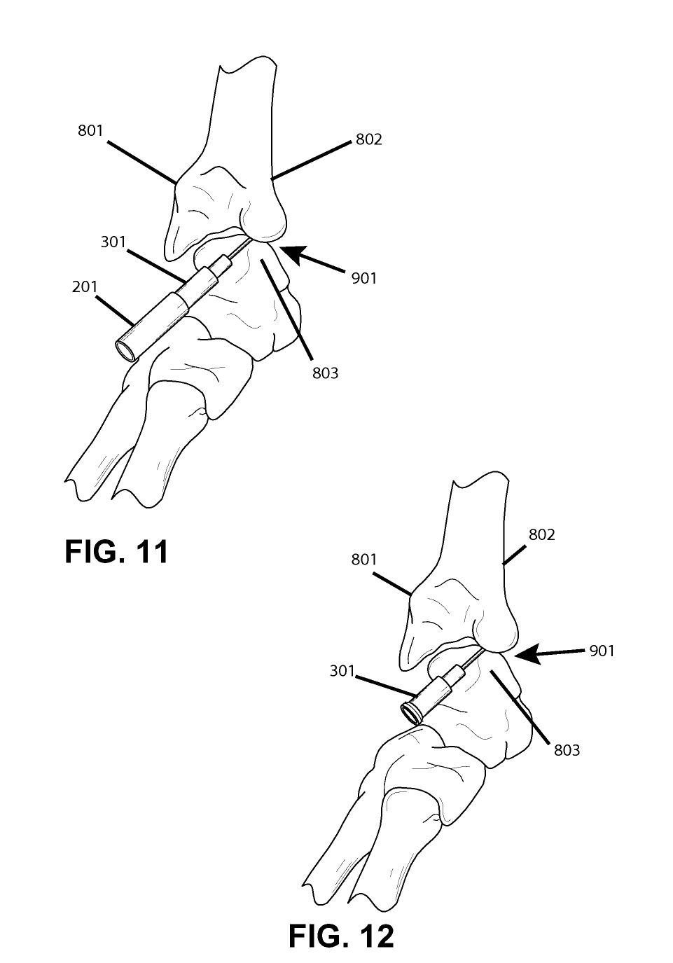

FIG. 11 shows angiocatheter localization within the anteromedial portal. Shown are stylet 201, angiocatheter 301, anteromedial port 901, fibula 801, tibia 802, and talus 803.

FIG. 12 shows the stylet removed. Shown are angiocatheter 301, anteromedial portal 901, fibula 801, tibia 802, and talus 803.

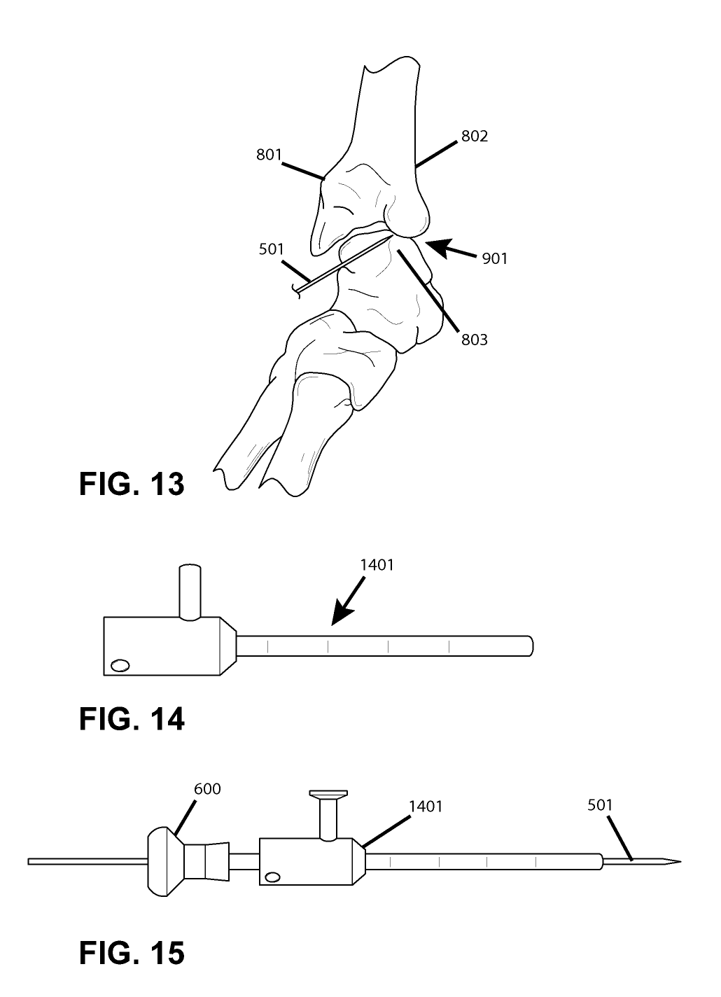

FIG. 13 shows a guide-wire inserted. Shown are guidewire 501, anteromedial portal 901, fibula 801, tibia 802, and talus 803.

FIG. 14 shows an arthroscopic sheath 1401.

FIG. 15 shows a guide wire 501 inside an arthroscopic joint surgery device 600 that is inside an arthroscopic sheath 1401.

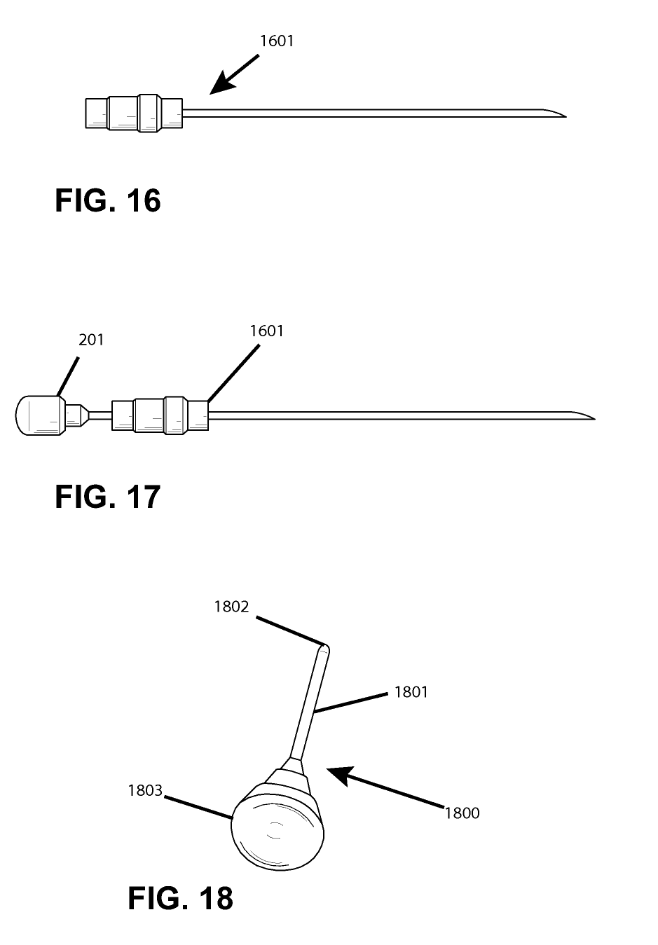

FIG. 16 shows a cannulated needle 1601.

FIG. 17 shows a stylet 201 inserted in a cannulated needle 1601 and is not secured to the hub.

FIG. 18 shows a perspective view of a standard obturator. Shown are a tube 1801, distal end 1802, and knob 1803. Note that there isn't a hole in the obturator 1800.

All patents and publications mentioned in the prior art are indicative of the levels of those skilled in the art to which the invention pertains. All patents and publications are herein incorporated by reference to the same extent as if each individual publication was specifically and individually indicated to be incorporated by reference, to the extent that they do not conflict with this disclosure.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations, and broad equivalent arrangements.

I claim:

1. An arthroscopic joint surgery device, the device comprising:
a tube measuring approximately 2.7 mm outer diameter, approximately 1.2 mm inner diameter, and approximately 85 mm length, and having a blunt rounded distal end or a sharp distal end and a blunt rounded proximal end; and
a cannulated blunt knob measuring approximately 15 mm outer diameter, approximately 1.2 mm inner diameter, and approximately 2.4 mm length, wherein the knob is attached to the proximal end of the tube and the inner diameter of the tube mates with the inner diameter of the knob.

2. The device of claim 1, wherein the joint portal is selected from the group consisting of an anterolateral ankle joint portal, anteromedial ankle joint portal, and posterolateral ankle joint portal.

3. The device of claim 1, wherein the knob is truncated, ridged, and configured for easy handling by a surgeon.

4. The device of claim 1, wherein the attachment between the tube and the knob is a threaded connection.

5. The device of claim 1, wherein the attachment between the knob and tube is further configured to enable the knob to be detached from the tube.

6. The device of claim 1, wherein the attachment between the knob and tube is permanent.

7. A method utilizing the device of claim 5, wherein the method comprises:
inserting an angiocatheter with a stylet or cannulated needle with a stylet into a joint portal under fluoroscopic guidance;
pulling the stylet out of the angiocatheter and inserting a guide-wire into the angiocatheter or cannulated needle under fluoroscopic guidance;
removing the angiocatheter or cannulated needle;
sliding the distal end of the tube over the guide-wire;
pushing the device into the joint portal under fluoroscopic guidance;
detaching the knob from the tube;
removing the knob and the guide-wire;
sliding an arthroscopic sheath over the tube into the joint portal under fluoroscopic guidance;
pulling out the tube;
inserting an arthroscope into the arthroscopic sheath under fluoroscopic guidance; and
beginning an arthroscopic procedure.

8. The method of claim 7, wherein the joint portal is selected from the group consisting of an anterolateral ankle joint portal, anteromedial ankle joint portal, and posterolateral ankle joint portal.

9. The method of claim 7, wherein the knob is truncated, ridged, and configured for easy handling by a surgeon.

10. The method of claim 7, wherein the attachment between the tube and the knob is a threaded connection.

11. A method utilizing the device of claim 6, wherein the method comprises:
inserting an angiocatheter with a stylet or cannulated needle with a stylet into a joint portal under fluoroscopic guidance;
pulling the stylet out of the angiocatheter and inserting a guide-wire into the angiocatheter or cannulated needle under fluoroscopic guidance;
removing the angiocatheter or cannulated needle;
placing the arthroscopic joint surgery device into an arthroscopic sheath;
sliding the distal end of the tube and arthroscopic sheath over the guide-wire into the joint portal;
removing the guide-wire;
inserting an arthroscope into the arthroscopic sheath under fluoroscopic guidance; and
beginning an arthroscopic procedure.

12. The method of claim 11, wherein the joint portal is selected from the group consisting of an anterolateral ankle joint portal, anteromedial ankle joint portal, and posterolateral ankle joint portal.

13. The method of claim 11, wherein the knob is truncated, ridged, and configured for easy handling by a surgeon.
